(19) United States
(12) Patent Application Publication
(10) Pub. No.: US 2014/0290334 A1
(43) Pub. Date: Oct. 2, 2014

(54) MEASUREMENT DEVICE AND METHOD FOR DETECTING THE HYDROCARBON CONTENT IN GASES

(71) Applicant: Beko Technologies GMBH, Neuss (DE)

(72) Inventor: Martin Friedrich, Loffingen (DE)

(73) Assignee: BEKO TECHNOLOGIES GMBH, Neuss (DE)

(21) Appl. No.: 14/353,469

(22) PCT Filed: Aug. 30, 2012
(86) PCT No.: PCT/EP2012/066812

§ 371 Date: Apr. 22, 2014

(30) Foreign Application Priority Data
Nov. 2, 2011 (DE) 102011055001.1

(51) Int. Cl. G01N 33/00

(52) U.S. Cl. CPC G01N 33/0047
USPC 73/23.2

(57) ABSTRACT

A measurement device and method for detecting the hydrocarbon content in gases includes: devices for subdividing an original gas stream into first and second measuring gas streams; first and reference gas units for producing first and second reference gas streams from a substream of the first and second measuring gas streams, respectively; two sensors for determining the content of hydrocarbon in the two measuring gas streams and generating a corresponding measurement results; and an evaluation unit for evaluating the measurement results of the two sensors. The first sensor is alternately fed the first measuring gas stream or the reference gas stream prepared by the first reference unit, and the second sensor is alternately fed the second measuring gas stream or the second reference gas stream prepared by the second reference gas unit. Because the two cycles are time-offset, a measurement value is continuously available.

### FIELD

The disclosure relates to a measurement device and a method for detecting the hydrocarbon content in gases.

### BACKGROUND

Conventional measurement devices are known with different sensor technologies and used to record the content of oil, hydrocarbons and oxidisable gases in air or compressed air, for example. Electrically heatable semiconductor oxide materials are frequently used, for example, which change their electrical resistance in the heated state, depending on the quantity of hydrocarbons contained in the air.

A further method is the detection of hydrocarbons by means of pellistors. For this purpose, the gas flow to be measured is conducted via a body made of heatable catalytic material, within which a heated platinum coil is located. The hydrocarbon concentration can be detected through the change in electrical resistance of the heated platinum coil and a second platinum coil which occurs due to the combustion heat of the hydrocarbon fraction at the catalyst.

The use of conventional flame ionisation detectors is used where hydrocarbons are combusted in a gas flow and the ionisation flow is measured between two electrodes in the flame.

A further method is the detection of the hydrocarbon concentration by means of ionisation. In this case the hydrocarbons are irradiated with ultraviolet light. The amount of energy in the light must be high enough in this case for electrons to be driven out of the hydrocarbon molecule. The number of these can be measured electronically.

The aforementioned methods are particularly suitable for the detection of higher concentrations in oxidisable gases, however the detection of lower concentrations in the low μg/m³ range or in the ppb range is not reliably possible.

The measured values generated by means of photoionisation detectors can only be used to infer the measured material quantity indirectly, as the measured values also depend on the molecular structure of the compound and vary quite significantly, even with the same total formulae. However, insofar as the compound being measured is constant, known and also uniform if possible, the concentration of the hydrocarbon fraction can be measured relatively reliably. However, the measuring accuracy drops as the concentration of hydrocarbons declines. In particular, the influence of the moisture content of the air rises in this case. As the hydrocarbon fraction decreases, the influence of the moisture in the air becomes greater and greater and measurements of hydrocarbon fractions cannot be conducted with sufficient accuracy in the lower mg/m³ range and particularly in the μg/m³ range.

For the different compressed air applications, different limit values are required for the oil fraction. Oil fractions consist of oil aerosols in droplet form and oil vapours. Oil aerosols and oil vapours may be partially or entirely eliminated from the compressed air flow by different methods.

WO 2010/094750 describes a measurement device and a method for detecting the hydrocarbon fraction in gases. An original gas flow is divided into a first gas flow and a second gas flow, both of which are analysed by a suitable measurement device. It is essential for the first gas flow to be conducted to the measurement device unchanged and for the second gas flow to be conducted in processed form. The hydrocarbon content is preferably determined via a signal difference between the first gas flow and the second gas flow in this case. Although the technology described allows even the smallest concentrations to be measured very reliably, the reliability of the technology is not yet adequate. Malfunctions affecting the measurement device or faults in the supply lines which can influence the measurement result are only identified after a long delay, if at all. Malfunctions or faults in the system can, moreover, only be identified and corrected at considerable expense, something that usually involves shutting down the entire plant.

### SUMMARY

The problem addressed by the disclosure is that of creating a measurement device for detecting the content of oil, hydrocarbons and oxidisable gases in gases, which on the one hand reliably measures even the smallest concentrations and, on the other hand, does not display the disadvantages of the state of the art. The measurement device should particularly exhibit a low error rate or allow measuring errors and/or malfunctions to be located easily and effectively. Furthermore, a problem addressed by the disclosure is that of providing a method of detecting the content of oil, hydrocarbon fractions and oxidisable gases in gases which is improved relative to the state of the art.

The problem is solved by a measurement device for detecting hydrocarbon fractions in gases comprising
mechanisms for dividing an original gas flow to be measured into a first measuring gas flow and a second measuring gas flow,
a first gas reference unit for producing a first reference gas flow from a partial flow of the first measuring gas flow,
a second reference gas unit for generating a second reference gas flow from a partial flow of the second measuring gas flow,
a first sensor for determining the hydrocarbon content in the first measuring gas flow and for producing a corresponding first measurement result,
a second sensor for determining the hydrocarbon content in the second measuring gas flow and for producing a corresponding second measurement result,
a sensor for measuring the volume flow,
an evaluation unit for evaluating the measurement results of the two sensors,
   wherein
the first sensor is alternately fed the first measuring gas flow or the first reference gas flow processed by the second reference gas unit,
the second sensor is alternately fed the second measuring gas flow or the second reference gas flow processed by the second reference gas unit.

Furthermore, the problem is solved by a method of detecting the hydrocarbon content in a gas flow which is characterized by the process steps:
division of an original gas flow to be measured into a first measuring gas flow and a second measuring gas flow,
production of a first reference gas flow from a partial flow of the first measuring gas flow,
production of a second reference gas flow from a partial flow of the second measuring gas flow,
alternate feeding of the first sensor with the first measuring gas flow or the first reference gas flow processed by the first reference gas unit,
alternate feeding of the second sensor with the second measuring gas flow or the second reference gas flow processed by the second reference gas unit,
determination of the hydrocarbon content in the first measuring gas flow and production of a corresponding first measurement result,
determination of the hydrocarbon content in the second measuring gas flow and production of a corresponding second measurement result,
evaluation of the measurement results from the two sensors.

According to the disclosure, the original gas flow to be measured is divided into a first measuring gas flow and a second measuring gas flow. The division of the original gas flow in this case may be achieved by an actual physical division by means of a separator, for example, alternatively the original gas flow may be fed alternately to the first sensor, to the second sensor or to the reference gas units in each case with the help of valves, for example. Catalysts, particularly oxidation catalysts, for example, can be used as reference gas units.

According to the disclosure, the measurement device has not only one, but two sensors for measuring hydrocarbon fractions, unlike in the known measurement devices. The two sensors are alternately exposed to reference gas or measuring gas. Both sensors determine their measurement result in this way, as the signal difference between the measuring gas and the reference gas produced, in other words the oxidised measuring gas, in each case. The measurement results may also contain different measured values, in addition to the hydrocarbon content.

The disclosure is based on the idea of providing at least two measuring gas paths and producing at least two measurement results accordingly. It is explicitly pointed out that the disclosure should not be defined based on the use of two sensors, but that further measuring gas paths and sensors are possible in addition. The disclosure is explained in the following simply by way of example for the use of two sensors.

At the start of the measurement, when starting the measurement device, for example, the sensors may be exposed to reference gas in a starting phase initially in both measuring gas paths. After a certain time the first gas path switches to measuring gas, while reference gas continues to be fed to the sensor in the second gas path. There is then a changeover, so that reference gas is fed to the first sensor in the first gas path and measuring gas is fed to the second sensor in the second gas path. This changeover is repeated any number of times, preferably in relatively short intervals of time. A duration of roughly 5 to 30 seconds per cycle, preferably 20 seconds per cycle, has proved adequate and reasonable.

It has proved particularly advantageous for the interval to be changed depending on the gas concentration. If there is a small concentration in the ppb range, the interval may preferably be 1 to 50 seconds, particularly approx. 30 seconds, whereas with large concentrations (e.g. 2 ppm) on the other hand it is roughly 80 to 150, preferably roughly 120 minutes.

The measurement device has an evaluation unit for evaluating the measurement results. In a first embodiment, the two sensor s may be connected to a single evaluation unit, although it is also conceivable for each sensor to be assigned its own evaluation unit. The evaluation unit has a processor which performs the necessary calculations. The individual gas flows or the total of all gas flows through the sensor or sensors may be measured or evaluated by the evaluation unit.

Furthermore, a display unit is provided, which displays the measurement results of the sensors or values calculated by the evaluation unit and/or further information. The display unit may also be advantageously configured as an input unit in the form of a touchscreen.

It is possible according to the disclosure for the two sensors to provide their measurement results for evaluation and/or display after a given sequence of steps. The display values represented by the display unit are therefore continuously present. The measurement result of the first sensor is shown initially, followed by the measurement result of the second sensor. The measuring cycles of the sensors are delayed in such a manner that a continuous measurement value signal is available.

It has proved particularly advantageous and beneficial to the user for the switching of the display values to be delayed in relation to the switching of the gas paths and exposure of the sensors. The measurement result of the first sensor can therefore still be seen on the display unit when measuring gas is already being applied to the second sensor and the measurement has begun. Only when a reliable measurement result is achieved for the second sensor is the indicator unit actuated by the evaluation unit and switched accordingly. A reliably measured measurement result is therefore constantly displayed. This is also particularly advantageous because the sensors usually require a certain stabilisation phase before they deliver a stable measurement result. The delayed display means that the stabilisation phase, during which the measurement result fluctuates comparatively sharply, is not visible to the user.

The use of two sensors also makes it possible, however, for these to be constantly offset against one another. If a deviation is identified, there may be various reasons for this. For example, the composition of the measuring gas may actually have changed over time. It is also possible, however, that one of the sensors is supplying incorrect measurement results. Finally, the gas paths may contain impurities or leaks.

According to the disclosure, the evaluation unit may conduct corresponding calculation operations and routines, in order to determine the cause of the deviation. If it emerges, for example, that after each change of sensors a deviation occurs, a change in the composition of the measuring gas can be virtually ruled out. In a particularly advantageous embodiment, both sensors are connected to two gas paths, so that in a following alignment it is possible to determine by switching gas paths whether the deviation is due to the gas paths or the sensors.

The evaluation unit advantageously runs a corresponding error analysis program independently and controls individual components of the measurement device, such as valves and/or throttles, for example, in order to perform an analysis of all elements of the gas paths and sensors. Due to the fact that each gas flow can be switched individually where necessary and measured at the outlet of the volume flow, very detailed error analyses are possible. It is also possible to present the cause of an error to the user via the display unit.

The evaluation unit is also capable of switching off one of the two defective sensors or gas paths independently, so that the measurement device continues to be ready for use. The evaluation unit advantageously provides corresponding information via the display unit, so that the measurement device can be repaired before there is a malfunction of the second gas path or sensor and the total failure associated with this. A substantial advantage results from the fact that the corresponding repair work can be carried out during a subsequent production break which is scheduled in any case.

Ultimately, the measurement device with a corresponding error analysis program is capable of analysing all components in the gas path independently. Flushing of the sensors with reference gas can also be initiated independently in regular cycles or due to a deviating or conspicuous measurement result from the measurement device.

The measurement result according to the disclosure may be furthermore configured such that both sensors can each be exposed to a calibrating gas from a storage bottle, for example. Apart from the unchanged measuring gas and the reference gas, a calibrating gas is therefore still routinely used to redefine the signal strength emerging from the sensor, for example. The calibrating gas (e.g. isobutene) has a defined hydrocarbon fraction, but no or only an exceptionally low moisture content. It is therefore possible according to the disclosure for the change in signal strength and measuring sensitivity to be reliably balanced by the ageing and contamination of the measurement device. The calibrating measurement may take place automatically at regular intervals, but it may also be initiated by the user at any time. In particular, it may be used within the framework of error analysis within the error analysis program. The data determined within the framework of the calibrating measurement is stored and can be called up at any time and used by the evaluation unit.

Moreover, it is possible through a balancing or comparison of the measurement results obtained with one another or with stored measurement results (from a reference database, for example), for calibration of the sensors to take place during exposure to measuring gas, reference gas or also calibrating gas. The term “measuring gas” in this case describes gas to be measured with an unknown ppm concentration, the term “reference gas” describes a known gas with a 0 ppm concentration and the term “calibrating gas” describes a known gas with a given ppm concentration.

Before the measurement device comes into operation, the two sensors may also be advantageously set by a corresponding supply of calibrating gas, in such a manner that it provides the same measurement result despite structural deviation or ageing.

Each measurement cycle may exhibit a calibrating phase according to the disclosure, in which measurement values currently measured by the individual sensor are analysed and compared.

A measurement cycle begins, for example, with the supply of reference gas. Following a stabilisation phase, a constant measurement result is supplied and the corresponding sensor is flushed with the reference gas. In the subsequent calibrating phase, the previously measured measurement results are compared, for example, with measurement results recorded for this sensor during production and the sensor is calibrated where necessary. Within the framework of the calibrating phase, further practical balances are also possible, for example with the measurement result from the other sensor during corresponding exposure to reference gas. A switch to measuring gas follows and a new stabilisation phase for a short period of time. In the subsequent stable operating state, in which a reliable measurement result can be provided, the measurement result is compared with the measurement result measured immediately before and/or with a stored measurement result from the other sensor and/or a further suitable reference value. The measurement cycle ends and the next measuring cycle begins with a switch to reference gas. The same measuring cycle takes place with a delay in the other gas path with the other sensor.

The evaluation unit may also constantly calculate a mean value for the measurement results from the two sensors and represent these via the display unit. Only when a preset deviation is exceeded does the measurement device react. For example, the error analysis program already described above is launched and/or only two measurement results are displayed alternately or simultaneously, preferably associated with an alarm. In a particularly simple embodiment, the mean value is still displayed and an alarm triggered.

The measurement device may be produced in a particularly cost-effective way when technical units in the form of modules are combined together. This may relate to valves, throttles, catalysts and sensors, for example. The corresponding elements and components are made of metal, for example.

In a particularly advantageous embodiment, the detection of hydrocarbon fractions is based on the principle of photoionisation and has a corresponding sensor unit with sampling probe. The sensor unit in this case is connected to the evaluation unit via a signal cable or wirelessly. The sampling probe may be advantageously mounted centrically from above in a riser, so that it can take gas centrally from the gas flow being monitored. The sensor unit has defined flow resistors which ensure a constant pressure and a constant volume flow of the individual measuring gases and are formed, for example, by a throttle with a defined bore or from a sintering metal. These are particularly low-maintenance and easy to clean. An alarm function is also provided which informs the user visually or acoustically in the event of pressure in the gas flows being too low or too high.

The flow rate of the different gas flows may be influenced by corresponding throttles, valves or flow rate reducers. These are preferably exchangeable and controllable in a particularly advantageous embodiment, so that the flow rate to the sensor can thereby be set on the one hand and, on the other, the desired mixing ratios of the gas flows being mixed can be reliably guaranteed.

Due to the fact that the valves in the gas path can be switched individually, it is also possible for reference air and measuring air to switch to the sensor simultaneously and dilute the measuring gas. The measuring range can thereby be extended upwards if the measuring air is extremely badly contaminated.

Customary oxidation catalysts can be used as reference gas producers, however other devices or methods of producing gases with the desired properties are also conceivable. Platinum-plated quartz wool is used as the oxidation catalyst, for example, which can easily be introduced into a container provided therefor. In a particularly advantageous variant, the reference gas producer is integrated into the measurement device, as a result of which only the different fluid or gas supplies need to be connected on site. The measurement device therefore has all connections for corresponding gas lines and also the electrical connection, so that it can be flexibly installed on site at any locations. In particular, the division of the measurement device according to the disclosure into the sensor unit with sensors, for example the sampling probe in the case of the photoionisation principle, and the evaluation unit with user surface (display) broadens the possibilities of a spatially flexible on-site installation still further. The evaluation unit with user surface is small in design and can be installed virtually anywhere, advantageously at an easily accessible position, while the slightly larger sensor unit can be arranged spatially separate from the evaluation unit at the measuring gas removal point.

The measuring device according to the disclosure may preferably be used with an oil-free compressing compressor to produce compressed air or compressed gas, although use with an oil-lubricated compressor is also conceivable, if there is a corresponding catalyst downstream of this. A bypass is preferably provided for maintenance work. The measurement device is also suitable in principle for further areas of application, for example for compressed gas bottles.

### BRIEF DESCRIPTION OF THE FIGURES

FIG. 1 shows a simplified functional representation of the measurement device according to the disclosure,

FIG. 2 shows a gas path circuit diagram for the measurement device,

FIG. 3 shows a measuring cycle diagram for the measurement device.

### DETAILED DESCRIPTION OF THE FIGURES

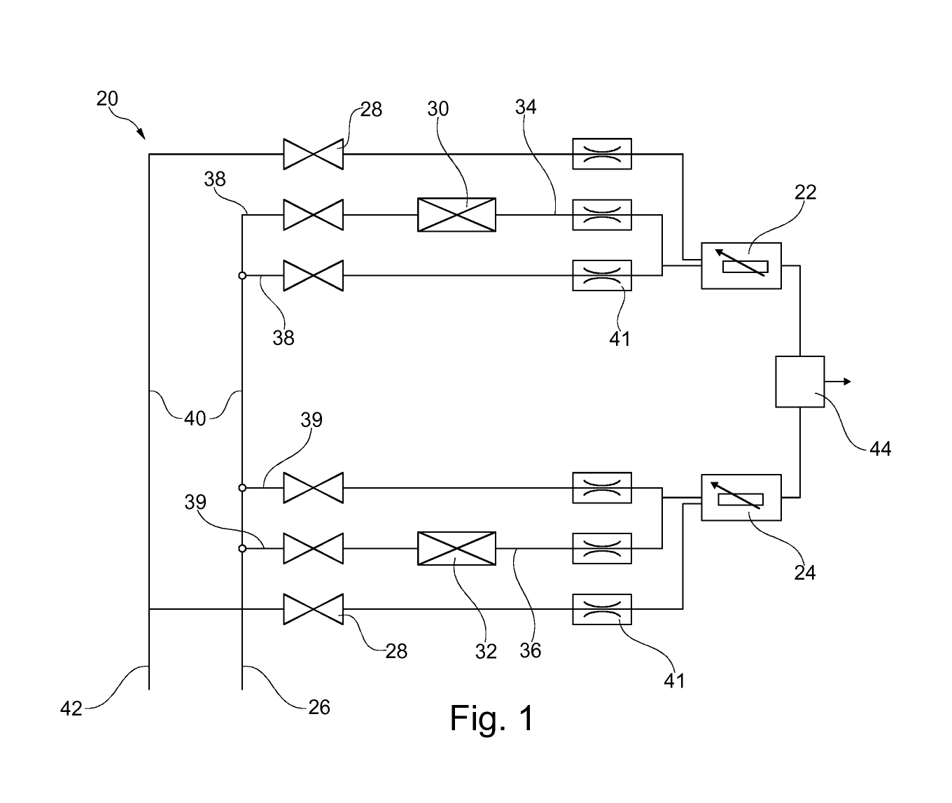

FIG. 1 shows the elements of a measurement device 20 according to the disclosure as a schematic representation. Said measurement device has two sensors, a first sensor 22 and a second sensor 24, preferably photoionisation sensors or metal oxide sensors.

An original gas flow 26 is divided via gas lines and with the help of valves 28 into a first measuring gas flow 38 and a second measuring gas flow 39. In the exemplary embodiment shown the original gas flow 26 is divided over time but a division into two separate volume flows is likewise possible.

The first measuring gas flow 38 is alternately fed partially to the sensor 22 or to a first reference gas unit and the second measuring gas flow 39 alternately partially to the second sensor 24 or a second reference gas unit 32. The reference gas units 30, 32 each produce a first reference gas flow 34 from the measuring gas flows 38, 39 or a second reference gas flow 36, which are then fed to the corresponding sensor 22, 24. The reference gas units 30, 32 are preferably configured as catalysts, particularly as oxidation catalysts.

The first measuring gas flow 38 (corresponds to the original gas flow 26) or the first reference gas flow 34 processed by the first reference gas unit is therefore fed alternately to the first sensor 22. The second measuring gas flow (39) (likewise corresponds to the original gas flow 26) or the second reference gas flow 36 processed by the second reference gas unit 32 is accordingly fed alternately to the second sensor 24.

The gas flows 26, 34, 36, 38, 39 are conducted through suitable gas lines 40 which form the corresponding gas paths. Throttles 41 are provided along the course of the gas lines 40, which throttle the gas flows 26, 34, 36, 38, 39 to a pressure level of 2 bar, for example, suitable for the sensors 22, 24 or the reference gas units 30, 32 (not shown, the throttle is only represented symbolically in FIG. 1). A corresponding throttle 41 may also be provided at the start of the gas line 40, as the original gas flow 26 is often introduced into the measurement device 20 at very high pressure. This first throttle 41 reduces the pressure level to 7 bar, for example.

Apart from the original gas flow 26, a calibrating gas flow is introduced into the measurement device 20 via a further gas line 40. This calibrating gas flow 42 is alternately fed either to the first sensor 22 or to the second sensor 24 without further processing, simply throttled.

The gas flows emerging from the sensors 22, 24 are combined via a device 44 and the total volume flow is measured. Through individual switching of the valves, the flow rate in each individual branch can thereby be measured.

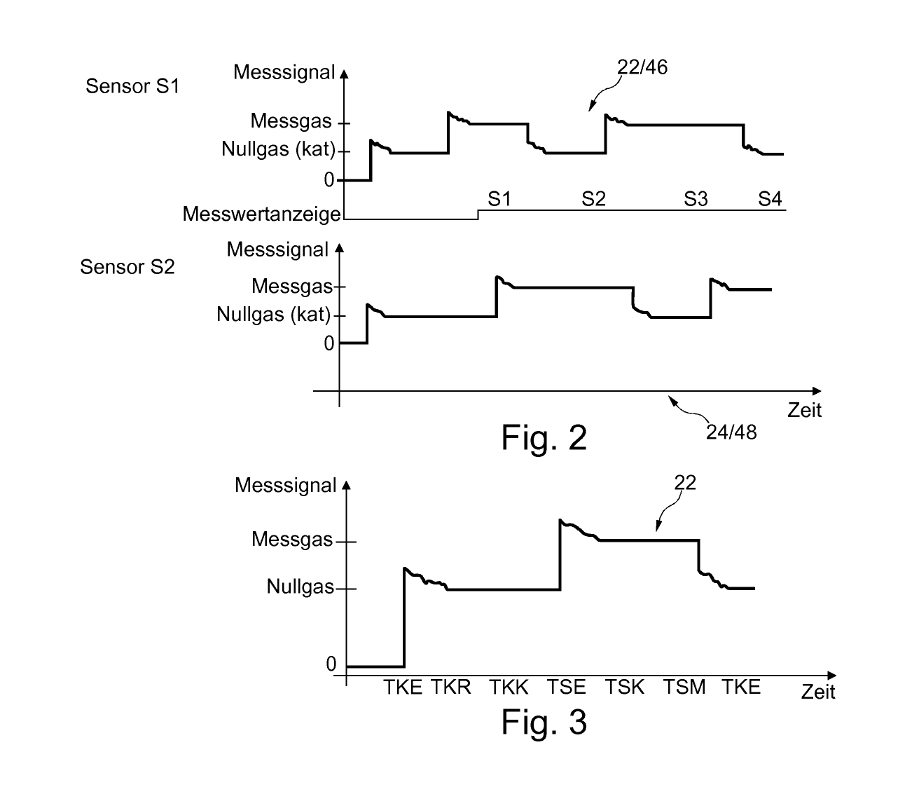

FIG. 2 illustrates in two gas flow circuit diagrams a possible method of operation of the measurement device 20. The first sensor 22 is assigned a first gas path circuit diagram 46 and the second sensor 24 a second gas path circuit diagram 48. The gas flows and a preferred measured value display over time are illustrated.

To begin with, when the measurement device 20 is started, for example, the sensors 22, 24 are initially exposed to the reference gas flow 34, 36 in each case in both gas paths during a start-up phase. After a certain time, the gas path of the first sensor 22 switches and the first sensor 22 is exposed to measuring gas. The sensor 22 conducts one or a plurality of measurements and produces a measurement result which is displayed as a display value S1. Reference gas is then fed once again to the first sensor 22. The same measuring cycle is conducted with a delay for the second sensor 24, which produces a display value S2.

It can be seen that the display values S1, S2 are displayed with a delay relative to the gas changeover of the sensors 22, 24. This means that the measurement device 20 or a corresponding display unit can display a display value continuously.

FIG. 3 illustrates in a measuring cycle diagram by way of example a measuring cycle of the first sensor 22. Initially after the first reference gas flow 34 is supplied, a transient oscillation of the signal is produced over time TKE. Flushing with reference gas takes place during a stable operating state TKR. In the subsequent calibration phase TKK, the measurement results obtained are compared with measurement results detected during production for this sensor 22 and calibrated where necessary. A balancing of the measurement results with measurement results of the same kind for the second sensor 24 is also conceivable.

Following a switchover to measuring gas, a new stabilisation phase begins over the time period TSE. During the following stable operating state TSK a calibration or balancing of the first sensor 22 with the measurement result for the second sensor 24 (TSM) measured immediately before takes place. The stabilisation phase may also be a delayed increase in the measured value, for example.

The TSM value of the sensor 22 and the sensor 24 is represented by the display unit.

Following a renewed switchover to the first reference gas flow 34, the measuring cycle starts afresh and is repeated continuously.

1. A measurement device for detecting the hydrocarbon fractions in gases, comprising
mechanisms for dividing an original gas flow to be measured into a first measuring gas flow and a second measuring gas flow,
a first gas reference unit for producing a first reference gas flow from a partial flow of the first measuring gas flow,
a second reference gas unit for generating a second reference gas flow from a partial flow of the second measuring gas flow,
a first sensor for determining the hydrocarbon content in the first measuring gas flow and for producing a corresponding first measurement result,
a second sensor for determining the hydrocarbon fraction in the second measuring gas flow and for producing a corresponding second measurement result, and
an evaluation unit for evaluating the measurement results from both sensors,
wherein
the first sensor is alternately fed the first measuring gas flow or the first reference gas flow processed by the second reference gas unit,
the second sensor is alternately fed the second measuring gas flow or the second reference gas flow processed by the second reference gas unit, and
the cycles of the sensors are delayed in such a manner that a continuous measurement signal is available.

2. The measurement device according to claim 1, wherein the first and second sensors are formed by photoionisation sensors.

3. The measurement device according to claim 1, wherein the first and second sensors are formed by metal oxide sensors.

4. The measurement device according to claim 1, wherein the reference gas units are configured as catalysts.

5. The measurement device according to claim 1, wherein along the course of the gas lines elements are provided from the group of throttles, valves, or flow rate reducers.

6. The measurement device according to claim 1, wherein a calibrating gas flow can be introduced into the measurement device via a further gas line.

7. The measurement device according to claim 6, wherein the calibrating gas flow is alternately fed to one of the first sensor and the second sensor.

8. The measurement device according to claim 1, wherein the reference gas unit is an integral component of the measurement device.

9. The measurement device according to claim 1, further including two spatially separate modules, a sensor unit with sensors and an evaluation unit with user surface.

10. A method of detecting the hydrocarbon fraction in a gas flow, including the following steps:
diving an original gas flow to be measured into a first measuring gas flow and a second measuring gas flow,
producing a first reference gas flow from a partial flow of the first measuring gas flow,
producing a second reference gas flow from a partial flow of the second measuring gas flow,
alternately feeding the first sensor with the first measuring gas flow or the first reference gas flow processed by the first reference gas unit,
alternately feeding the second sensor with the second measuring gas flow or the second reference gas flow processed by the second reference gas unit,
determining the hydrocarbon fraction in the first measuring gas flow and production of a corresponding first measurement result,
determining the hydrocarbon fraction in the second measuring gas flow and production of a corresponding second measurement result,
evaluating the measurement results from both sensors,
wherein
at the start the sensors are exposed to the reference gas flow in each case in a starting phase initially in both gas paths,
after a certain time the first sensor is exposed to measuring gas and the first sensor conducts at least one measurement and produces a measurement result which is displayed as a display value,
reference as is the fed once again to the first sensor and
the same measuring cycle is conducted with a delay for the second sensor, which produces a display value.

11. The method according to claim 10, wherein a plurality of display values are displayed in a delayed manner relative to the gas switch of the sensors.

12. (canceled)

13. The method according to claim 10, wherein apart from the unchanged measuring gas and the reference gas, a calibrating gas is still routinely supplied.

14. The method according to claim 10, wherein the measuring interval can be changed depending on the gas concentration; if there is a small concentration in the ppb range, a measuring interval lasts 1 to 50 seconds, particularly approx. 30 seconds, with large concentrations in the ppm range, on the other hand, it is roughly 80 to 150 minutes.

15. The method according to claim 10, wherein the switching of the display values is delayed relative to the switching of the gas paths and the exposure of the sensors.

16. The measurement device according to claim 1, wherein the reference gas units are configured as oxidation catalysts.

17. The method according to claim 10, wherein the measuring interval can be changed depending on the gas concentration; if there is a small concentration in the ppb range, a measuring interval lasts approximately 30 seconds and if there is a large concentration in the ppm range, a measuring interval lasts approximately 120 minutes.
